(19) United States
(12) Patent Application Publication
(10) Pub. No.: US 2016/0201020 A1
(43) Pub. Date: Jul. 14, 2016

(54) PHOTOBIOREACTOR WITH LATERALLY LIGHT-EMITTING LIGHT CONDUCTOR MATS

(71) Applicant: Airbus Defence and Space GmbH, Ottobrunn (DE)

(72) Inventors: Johann Göbel, Munchen (DE); Robert SCHREIBER, Grafelfing (DE); Jennifer WAGNER, Hamburg (DE)

(21) Appl. No.: 15/074,584

(22) Filed: Mar. 18, 2016

Related U.S. Application Data
(63) Continuation of application No. PCT/DE2014/000464, filed on Sep. 3, 2014.

(30) Foreign Application Priority Data
Sep. 18, 2013 (DE) 10 2013 015 423.5

(51) Int. Cl. C12M 1/00

(52) U.S. Cl. CPC C12M 21/02

(57) ABSTRACT

A photobioreactor and a photobioreactor system are proposed in order to cultivate phototrophic organisms for the purpose of generating fuels, for example. The photobioreactor comprises a container and at least one laterally light out-coupling light conductor mat. The phototrophic organisms are received in the container together with a nutrient solution. One or preferably more light conductor mats are arranged within the container and each comprises a plurality of light conducting fibres which are arranged and/or designed such that light which is coupled into a fibre at one end of the fibre leaves the fibre laterally at least in part. A large adjacent volume within the container can thus be extensively illuminated by means of the light conductor mat, in order to thus increase efficiency of the photobioreactor. A photodetector that is externally coupled to the fibres can allow on-site monitoring of vital functions of the organisms.

### CROSS-REFERENCE TO RELATED APPLICATIONS

This is a continuation of International Application No. PCT/DE2014/000464, filed Sep. 3, 2014, which application claims priority to German Application No. 10 2013 015 423.5, filed Sep. 18, 2013, which are hereby incorporated by reference in their entirety.

### TECHNICAL FIELD

This relates to a photobioreactor for cultivating phototrophic organisms.

### BACKGROUND

Phototrophic organisms are the smallest of living organisms; for example, in the form of micro-organisms, which can directly use light as an energy source for their metabolism. Phototrophic organisms include, for example, certain plants, mosses, microalgae, macroalgae, cyanobacteria, and purple bacteria.

For different purposes, it may be desirable to produce biomass, for example, in the form of algae, in large quantities and in a cost-effective manner. For example, biomass of this type may be used to produce alternative biofuels, e.g. for the transport sector.

In order for it to be possible to produce biomass on an industrial scale, what are known as bioreactors are used. A bioreactor is a system for producing organisms outside their natural environment and within an artificial technical environment. Photobioreactors are used to cultivate phototrophic organisms. A photobioreactor provides the phototrophic organisms both with light and also with CO₂, and, if necessary, with a suitable nutrient solution, so that these organisms can accordingly synthesise biomass.

Generally, both open and closed systems are known for photobioreactors. Each of these types of photobioreactors has certain advantages and drawbacks.

In the case of open photobioreactor systems, sometimes also referred to as open ponds, phototrophic organisms are bred in open basins or pools in a controlled manner. Here, a nutrient solution or culture suspension that contains all the nutrients and CO₂ required for the organism in question is usually fed into a circuit and is usually directly illuminated by the sun from the open surface.

Potential advantages of such open photobioreactor systems are comparatively low technical complexity and low electric power consumption.

However, illumination solely via the surface that is open at the top means that only low volumes can be supplied with sufficient light. Light can usually only penetrate a few centimetres in depth into a nutrient solution to which organisms have been added. The depth of such open photobioreactor systems is therefore generally limited to 20 to 30 cm. The low average light input leads to low area-related growth rates. A high surface area therefore needs to be provided for open photobioreactor systems. As a result, the costs of such photobioreactors are considerably increased, in particular in densely populated regions.

In addition, a high level of evaporation and thus effects involving an increase in salinity may occur on the open surface. A significant amount of CO₂ may furthermore diffuse into the atmosphere via the open surface. Conversely, contaminants may get into an open photobioreactor via the open surface, contaminate the photobioreactor and thus compromise the purity of the product. Furthermore, any heating or cooling that is potentially required for such open photobioreactor system proves difficult. When the system is solely illuminated by sunlight, depends on daylight hours, with deeper layers often only being insufficiently illuminated, while very high illumination intensities may occur directly on the surface of the open system, which potentially could lead to what is known as photoinhibition.

When considering the above-mentioned drawbacks in conjunction with limiting boundary conditions, this may in particular mean that open photobioreactor systems in the form of open ponds can be used all year round only in very specific geographical regions.

In order to reduce the influence of environmental conditions and also to achieve a higher yield when cultivating phototrophic organisms, closed photobioreactor systems have been developed. In such closed systems, a nutrient solution is fed through a closed circuit together with the organisms, and is usually illuminated from the outside during the process.

For example, in a tube photobioreactor, glass tubes or plastics tubes are joined together to form a closed circuit, and nutrients and CO₂ are supplied to the organisms enclosed therein by means of a central unit, which for example may contain suitable pumps and sensors.

Closed photobioreactors generally allow for a high level of process control, since the organisms and the surrounding nutrient solution can be effectively heated and/or cooled in the closed system, a pH value can be monitored and adjusted if necessary and additional light can be provided. Closed systems allow a high level of productivity with low surface area requirements, since for example a plurality of closed systems can be arranged one above the other or tubes of a system can extend in a vertical direction and can be illuminated from all sides in the process. Here, however, shadow effects always have to be taken into account. In addition, high product purity is possible, with there being a low amount of contamination, a low level of evaporation, and low electromagnetic interference (EMC).

However, the technical complexity and the corresponding investment costs for the system are generally very high when constructing complex closed photobioreactors compared with open systems.

A large number of technical solutions have already been developed in order to increase the efficiency of photobioreactors. As a measure of the efficiency of a photobioreactor, the amount of resources required, such as energy to be provided in the form of light and/or electricity, the surface area to be provided, the nutrients to be provided, etc., can be understood in relation to the yield of the photobioreactor in the form of biomass having the highest possible amounts of energy chemically stored therein.

For example, a photobioreactor having rotationally oscillating light sources has been described in EP 2 520 642 A1.

### SUMMARY

There exists a need to providing a photobioreactor for cultivating phototrophic organisms which allows high efficiency together with low investment costs for the system and/or low operating costs.

According to an aspect of an embodiment, a photobioreactor is proposed which comprises a container and at least one laterally light out-coupling (or light emitting) light conductor mat. The container is designed to receive phototrophic organisms together with a nutrient solution. The light conductor mat is arranged within the container and comprises a plurality of light conducting fibres, which are arranged and/or designed such that light that is coupled into the fibres at one end of a fibre leaves the fibres laterally at least in part.

Concepts for of the photobioreactor according to the embodiment may inter alia be considered to be based on the following ideas and knowledge: phototrophic organisms should be optimally supplied with light and nutrients in order for them to breed. However, light may only propagate over very short distances of a few centimetres, in particular in a nutrient solution to which large numbers of organisms have been added. A photobioreactor in which the nutrient solution is received in a container and the container is only illuminated from outside therefore has to provide, in a comparatively small volume, the largest possible outer surface area that can be illuminated. This reassures a large base area or floor area to be available for the photobioreactor, for example as in an open-pond system, or the necessity for a complex structural design, as in conventional closed systems such as tube photobioreactors.

Accordingly, embodiments of the photobioreactor are now proposed that relate to arranging one or more special light conductor mats in a container receiving the nutrient solution. Here, the light conductor mat is specifically designed not only to couple out or emit light that is coupled into the ends of the fibres forming the light conductor mats at opposite ends of the fibres, but also to couple out the light laterally, that is to say transversely to a surface of the light conductor mat. The light can be coupled out as homogeneously as possible over the entire surface area of the light conductor mat. This means that large amounts of light can be introduced into the inside of the container of the photobioreactor so as to be largely homogeneously distributed over the surface of the light conductor mat. By using the at least one laterally light out-coupling light conductor mat, high efficiency and potentially other advantages that are described in greater detail below can thus be achieved for the proposed photobioreactor.

According to an embodiment, in a photobioreactor the light conductor mat can be arranged such that a minimum distance between a position in the container and the closest region of the light conductor mat is less than 10 cm, preferably approximately 5 cm, for at least 90% of the possible positions within the container.

In other words, the light conductor mat can be designed and arranged in the container such that, in the majority of the volume of the container, each point is less than 10 cm, preferably less than 5 cm, away from the closest region of the light conductor mat, and thus light that is coupled out of the light conductor mat from this point can even be obtained through a murky nutrient solution. In this way, significant portions of the volume of the container can be efficiently supplied with light without needing the container to have a very large surface area relative to the volume received therein.

In particular, according to one embodiment, the container may have dimensions of greater than 50 cm, preferably greater than 100 cm, in all directions in space.

In other words, the container of the photobioreactor may have a large volume relative to its outer surface area. In particular, the container may have dimensions in all directions in space which are considerably greater than a typically predominant penetration depth of light in a photobioreactor nutrient solution to which organisms have been added.

According to an embodiment, instead of a single light conductor mat, the photobioreactor may also have a plurality of laterally light out-coupling light conductor mats, which are distributed over the entire volume of the container. In this case, the light conductor mats can be distributed as evenly and homogeneously as possible over the entire container volume, so that light can be coupled in and distributed evenly over the entire container volume.

According to an embodiment, the light conducting fibres are arranged in the light conductor mat so as to be locally bent such that some of the light guided in a fibre is locally coupled out of the fibre laterally, at least in regions having a minimum radius of curvature.

A suitable local curvature of the fibres in the light conductor mat makes it possible for the light guided therein to no longer be totally internally reflected on the surface of a fibre, but rather to be laterally coupled out of the fibre at least in part. For example, the light conducting fibres may be arranged in the light conductor mat such that sufficiently locally bent regions are produced and a plurality of these sufficiently locally bent regions are distributed as evenly as possible over the surface of the light conductor mat.

According to an embodiment, the light conducting fibres are woven in the light conductor mat. By weaving light conducting fibres, a woven fabric having regular structures and in particular having regular sufficiently locally bent regions can be produced.

According to an embodiment, the light conducting fibres may have local variations in the index of refraction. Such local variations in the index of refraction may be produced in different ways, for example by locally providing indentations, notches, by local melting or in the form of a laser grating. The local variations in the index of refraction may in particular be produced at a plurality of positions in the longitudinal direction of the light conducting fibres and may be located close to the surface of said fibres, or deep within the volume of the fibres. At such local variations in the index of refraction, light propagating in the light conducting fibre may be appropriately refracted such that it leaves the fibre laterally. By appropriately distributing such local variations in the index of refraction across the fibres and thus across the entire light conductor mat, light can be appropriately laterally coupled out of the light conductor mat as homogeneously as possible over an entire side face (lateral area) of the light conductor mat.

Alternatively or additionally, according to an embodiment, scattering centres and/or fluorescence centres may be integrated in the light conducting fibres. Such scattering centres or fluorescence centres may be embedded in the light conducting fibre volume in the form of small particles that are of a suitable size and made of a suitable material, or in the form of quantum dots, and this means that light guided in the fibres is scattered at the scattering centres and/or generates fluorescent light at the fluorescence centres, which can then leave the fibres laterally.

According to an embodiment, the light conducting fibres are made of a material which substantially does not transmit light in the infrared wavelength range. Here, the infrared wavelength range can be considered a wavelength range of above 800 nm. “Substantially does not transmit” can be understood to mean that less than 30%, preferably less than 10%, of an infrared component of light that is coupled into an end of a fibre is transmitted into the inside of the container by means of the fibres. Most phototrophic organisms cannot use infrared light for growth or metabolism. By using light conducting fibres that do not transmit in the infrared range for the light conducting mat, these light components that are not required for the growth of the organisms can be prevented from reaching the internal volume of the photobioreactor and from causing said photobioreactor to heat up considerably, which otherwise would have to be counteracted by appropriate cooling measures.

According to an embodiment, the photobioreactor further comprises a mat moving device, which is designed to move the at least one light conductor mat relative to the container. A light conductor mat that is moved by a mat moving device may be used here to continuously move or circulate the nutrient solution received in the container of the photobioreactor. In this way, it can be ensured that nutrients and phototrophic organisms are continuously mixed, and this brings about better growth of the organisms. In this case, the light conductor mat can be moved by the mat moving device preferably transversely to the surface thereof, for example in a translational, rotational, vibrating or oscillating manner.

A movement may in particular be made periodically. By using the option of actively moving the light conductor mat within the nutrient solution, a separate stirrer that is usually used in conventional photobioreactors can be omitted.

According to a further aspect, a photobioreactor system is proposed which comprises a photobioreactor and a light source. In this case, the light source is coupled to light conducting fibres of the at least one light conductor mat of the photobioreactor in order to couple light from the light source into the light conducting fibres.

According to an embodiment, the light source may be designed to collect sunlight and couple it into the light conducting fibres. The light source may for example be in the form of suitable collectors or mirrors, by means of which sunlight is focused on and/or directed to ends of the light conducting fibres of a light conductor mat and is coupled into the light conducting fibres in this way. Natural sunlight can thus be used in order to efficiently and substantially evenly illuminate an internal volume of the photobioreactor by means of the light conductor mat.

Sunlight can be collected in several ways. Light conductor mats which are located outside the container (and which may be structurally similar to those inside the container) may be used for absorbing and coupling light into the light conductor mats located inside the container, for example. Here, there is the option of orienting the external absorbing light conductor mats to the light according to the position of the sun by means of a simple device in order to make it possible to optimally couple in the light.

Alternatively or additionally, according to an embodiment, the light source can be designed to artificially generate light and couple it into the light conducting fibres. The light to be coupled in may in this case be generated by lamps, LEDs, a laser or other technical means. Alternatively or additionally providing such technical light sources for generating artificial light may, by contrast with the use of only sunlight, allow the system to be independent of the daylight rhythm. In addition, artificial light can be deliberately generated to have suitable properties. For example, the artificial light can be generated in a pulsed or intermittent manner, as a result of which the photosynthetic efficiency of phototrophic organisms can be significantly increased. The artificial light may also be generated to have a low infrared component, in order to prevent unnecessary heating within the photobioreactor.

In particular, according to an embodiment, the light source may be designed to only couple light that is substantially within a wavelength range of from 400 to 700 nm into the light conducting fibres. In this case, “substantially” can mean that at least 70%, preferably 90%, of the light energy coupled in is within the specified wavelength range. The fact that light is predominantly coupled into the light conducting fibres in the wavelength range can be achieved either by the light source itself mainly generating light in the wavelength range or by the light source indeed generating light having a broader spectrum, but then undesired spectral ranges being sorted out, for example by means of filters, and not being coupled into the light conducting fibres. Light in the wavelength range has proven to be particularly favourable for growth of phototrophic organisms and should therefore preferably be radiated into the inside of the photobioreactor by means of the light conductor mat.

According to an embodiment, the photobioreactor system may further comprise a photodetector which is connected to light conducting fibres of the at least one light conductor mat of the photobioreactor in order to collect light which has been coupled into the light conducting fibres from the inside of the container of the photobioreactor.

In this embodiment, advantage may be taken of the fact that not only can light be coupled into the inside of the photobioreactor by the light conducting fibres of the light conductor mat from the outside, but also, vice versa, light which has been stimulated inside the bioreactor can be conveyed to the outside by the light conducting fibres and can be detected by one or more photodetectors at this point. Many phototrophic organisms react to stimulation by emitting light, and therefore by detecting light emitted inside the container of the photobioreactor, conclusions can be drawn about the vital functions of the organisms to be cultivated. In particular, by making it possible to couple light emitted by the organisms into fibres of the light conductor mat laterally and thus by it preferably being possible to absorb the light along an entire lateral surface of the light conductor mat and to pass the light to the photodetector, on-site monitoring of vital functions of the organisms received inside the photobioreactor is possible across very large volume ranges of the entire container.

Furthermore, the optical density which is in direct correlation with the cell density in the cultivation medium can be determined on-site by means of the light conductor mats and the photodetector. For this purpose, light of a particular wavelength is introduced by means of a light conductor mat and the intensity of the emitted light is transmitted to the photodetector by means of an adjacent spaced-apart light conductor mat.

It is noted that possible advantages and features of embodiments are described herein sometimes with reference to a photobioreactor and sometimes with reference to a photobioreactor system. A person skilled in the art would recognise that the various features can be combined or replaced in an appropriate manner in order to arrive at further embodiments.

### BRIEF DESCRIPTION OF THE DRAWINGS

In the following, embodiments are described with reference to the accompanying drawings, and neither the description nor the drawings should be interpreted as having a limiting effect.

FIG. 1 shows a photobioreactor according to an embodiment.

FIG. 2 shows a detail of a light conductor mat for a photobioreactor.

FIG. 3 shows a detail of an alternative light conductor mat for a photobioreactor.

FIG. 4 shows a detail of a light conductor mat for a photobioreactor.

FIG. 5 shows a photobioreactor system according to an embodiment.

The figures are merely schematic and are not to scale. In the different figures, like reference numerals denote like or functionally like features.

### DETAILED DESCRIPTION

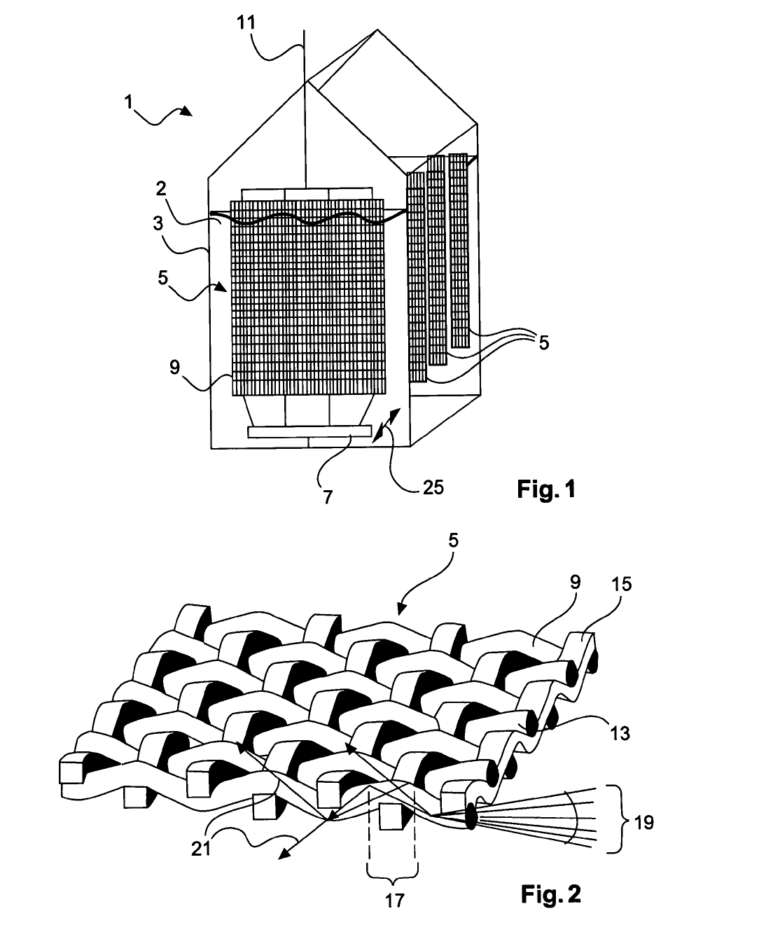

FIG. 1 is a schematic perspective view of a photobioreactor 1 according to an embodiment. The photobioreactor 1 comprises a container 3, in which phototrophic organisms can be received together with a nutrient solution 2. A plurality of light conductor mats 5 are arranged in the container 3 so as to be approximately in parallel and spaced apart from each other. Each of the light conductor mats 5 is designed to have a plurality of light conducting fibres 9 which are arranged and designed such that light, which for example is coupled into ends of the fibres 9 by means of a shared light guide 11 that is led out of the container 3, leaves the fibres 9 laterally at least in part, and thus transversely to the surface of the light conductor mats 5.

The container 3 may have any desired geometry. For example, as shown in FIG. 1, the container may be a rectangular cuboid or square cuboid. Alternatively, the container 3 may also be cylindrical, spherical or may have another shape.

Here, the container 3 may have an appropriate geometry in which a large volume can be received in the container 3 at the same time as there being a comparatively small surface area. In particular, the depth of the container 3 may be greater than the lateral dimensions and/or the base area of the container 3. Here, the depth of the container 3 is intended to be measured in a direction transverse to a main extension plane of the light conductor mat. In particular, the container 3 may have dimensions of greater than 50 cm, preferably greater than 1 m, in all directions in space, i.e. in height, width and depth.

The container is intended to be tightly sealed at least in a lower region, so that liquid nutrient solution, together with the phototrophic organisms received therein, can be held in the container 3. As shown in FIG. 1, the container 3 may also be closed and tightly sealed in an upper region, so that an intrinsically closed photobioreactor is formed. Alternatively, the container 3 may however also be open at the top, in order to form an open photobioreactor. Walls of the photobioreactor 1 (these only being outlined in FIG. 1 for better illustration, in order to make it possible to see internal components of the photobioreactor) may be made of any fluid-tight materials, such as a plastics material or metal, and do not necessarily need to be translucent.

Each of the light conductor mats 5 may be made up of a plurality of light conducting fibres 9. In this case, the light conducting fibres may be rigidly or loosely interconnected in different ways. The light conductor mat may for example be provided in the form of a woven fabric, a knitted fabric, a non-woven fabric or another three-dimensional structure, for example a honeycomb structure. In this case, the light conductor mat is preferably planar for example, it being possible for a thickness transverse to the main extension direction of the surface to be less than 10 mm, preferably less than 2 mm. The light conductor mat is intrinsically flexible and pliable and in this respect has similar mechanical properties to a film. However, the light conductor mat is permeable to fluid as it is made up of a plurality of fibres, that is to say that fluid, for example in the form of the nutrient solution, can slowly flow through the light conductor mat.

The fibres 9 forming the light conductor mat 5 guide light effectively at least in the interior thereof, that is to say in a core, i.e. they have high optical transparency. The fibres may be made of transparent materials such as glass or a transparent plastics material, in particular a transparent polymer such as polymethyl methacrylate (PMMA). The fibres 9 or cores of the fibres 9 may have diameters in the range of a few micrometres up to a few millimetres. Typical diameters are in the range of from 5 to 2 mm, in particular of from 5 to 30 μm. Each of the fibres 9 may be very pliable and for example may be bent in radii of curvature of less than 10 mm.

In order for it to be possible to guide light in the interior of the fibre 9, the fibre 9 may be sheathed with a layer referred to as “cladding”, which has a lower optical index of refraction than a material in the core of the fibre 9. At shallow angles, light incident on such cladding is guided back into the core of the fibre by total internal reflection and can thus propagate in an elongate fibre over large distances.

However, for the specific use of light conductor mats in a photobioreactor, it is also considered possible to provide light conducting fibres without such cladding, since it is assumed that the nutrient solution surrounding the individual fibres should likewise have an appropriate optical index of refraction, so that the desired total internal reflection takes place.

The light conducting fibres may be designed to have the smoothest possible surface, in order for example to prevent deposits or dirt from accumulating on individual fibres. The fibres may optionally be hydrophobically coated, for example covered with a layer of titanium dioxide (TiO₂). A coating comprising a material that increases scratch resistance may also be provided. Potential coatings may be applied for example using plasma processes, a sol-gel technique or by varnishing.

As explained in greater detail below on the basis of specific embodiments, the light conductor mats 5 and the light conducting fibres 9 used therein are designed such that light guided in the fibres 9 is coupled out laterally at least in part, that is to say transversely to a surface of the light conductor mat 5. In this case, the portion of the light that leaves laterally is intended to be significant in relation to the total amount of light leaving the fibres 9 of the light conductor mat 5, for example at least 10%, but preferably at least 50%, potentially even at least 90%. A portion of light that leaves the light conductor mat 5 laterally may in this case preferably leave the light conductor mat laterally such that it is homogeneously distributed over the light conductor mat. In other words, the light coupled into an individual fibre may leave the fibre laterally such that it is distributed over the entire length of the fibres as far as possible.

FIG. 2 shows an embodiment of a light conductor mat 5 in which a plurality of light conducting fibres 9 are woven to form a woven fabric. The fibres of the woven fabric may be interwoven in various weave patterns in this case. Either just warp threads 13 that extend in the longitudinal direction or just weft threads 15 that extend in the transverse direction, or both warp threads 13 and weft threads 15, may be designed as light conducting fibres 9.

Owing to the woven structure, the light conducting fibres 9 are locally bent such that, at least in regions 17 having a minimum radius of curvature, some of the light 19 coupled into a fibre and guided therein in the longitudinal direction of the fibre is coupled out of the fibre 9 laterally. Here, the portions 21 of light that are coupled out are emitted transversely to the direction of extension of the light conductor mat 5 and may thus illuminate adjacent volumes within the container 3 of the photobioreactor 1.

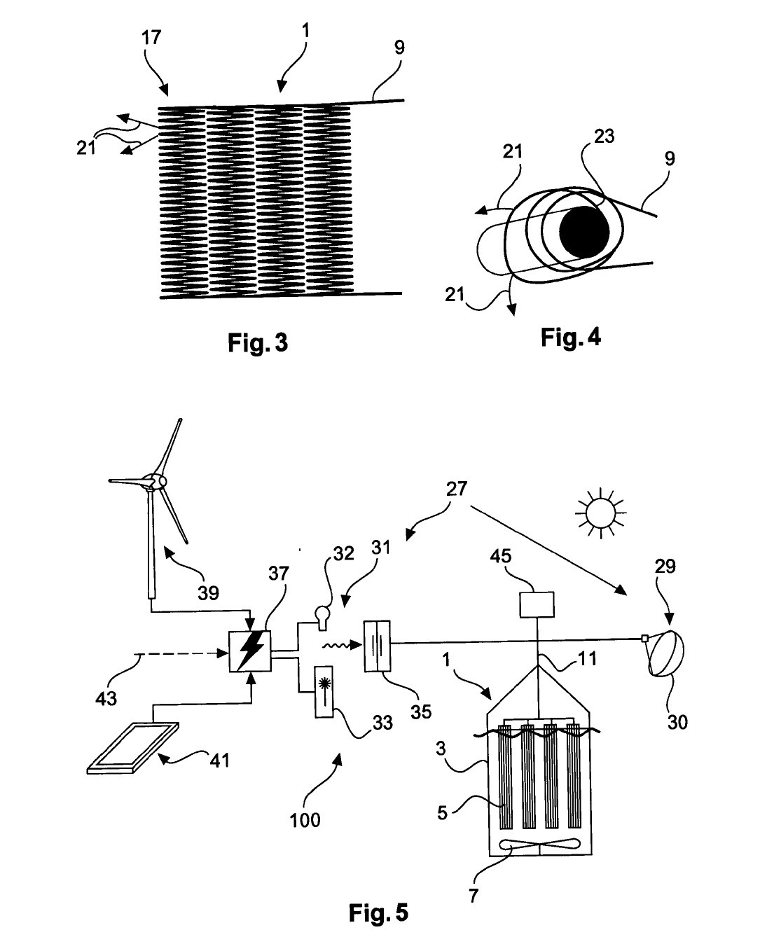

FIG. 3 shows an alternative embodiment of a light conductor mat 5. A plurality of light conducting fibres 9 are laid in this light conductor mat 5 in a serpentine manner, so that local light out-coupling 21 takes place in heavily bent regions 17.

FIG. 4 shows another alternative embodiment of how portions 21 of light can be laterally coupled out by means of light conducting fibres 9. In the example shown, the fibre 9 is wound, at a small radius, around a core medium 23, which for example may in turn be a fibre, so that total internal reflection is locally prevented within the fibre 9 owing to the small radius of curvature and therefore portions 21 of light are coupled out laterally.

Light may also be laterally coupled out of individual light conducting fibres 9 by there being local variations in the index of refraction in the light conducting fibres 9. In other words, the fibres 9 are produced or processed such that light which propagates within the fibres along their length passes through regions having different indices of refraction or impinges on such regions.

In this case, the variations in the index of refraction may be provided only on the surface of a fibre, or alternatively may also extend into the internal volume of the fibre.

For example, the outer surface of a fibre may be ground, notched, indented or similar so that the desired variations in the index of refraction are produced in the region of these changes in the shape of the fibres. Here, cladding provided on a surface of the fibre may be locally removed if necessary, and this further promotes the lateral out-coupling of portions of light.

Alternatively, a fibre density may be locally altered by means of a laser by temporary local heating, for example, which is also referred to as laser grating or fibre grating. In this process, an outer surface of the fibre does not need to be modified, and in particular does not need to be altered in terms of geometry, and can remain smooth, so that there is no risk of local dirt accumulation. Similar effects may be achieved by locally melting the surface of a fibre, in particular if these are polymer fibres.

Another option for locally coupling out portions of light laterally may be implemented by embedding microscopically small scattering centres or fluorescence centres in light conducting fibres 9. Scattering centres may be minute or infinitesimal particles of preferably highly optically reflecting material, for example the smallest of metal particles. Fluorescence centres may for example be particles made of a fluorescent material.

As shown in FIG. 1, a plurality of light conductor mats 5 may be arranged inside the container 3 of a photobioreactor 1 such that they are evenly distributed over the entire volume of the container 3. In this case, the light conductor mats 5 extend in approximately parallel planes with respect to one another, for example in parallel with planes of side walls of the container 3. A distance between adjacent light conductor mats 5 may preferably be less than 20 cm in this case, so that any point inside the container 3 is at most 10 cm away from one of the light conductor mats 5 over large regions of the container 3. In this way, preferably the entire volume of the nutrient solution received in the container 3, or at least large portions thereof, can be evenly supplied with light which has been introduced into the container 3 by the shared light guide 11 and has then been radiated into the nutrient solution by being laterally coupled out of the light conductor mats 5.

A mat-moving device 7 is further provided in the container 3 of the photobioreactor 1. This mat-moving device 7 comprises its own drive and is designed to move each of the light conductor mats 5 transversely to its main direction of extension, that is to say in the direction of the arrow 25. As an alternative to such translational movement, rotational movement or any other type of movement may also be carried out. In particular, the movement may be carried out periodically, for example in an oscillating or vibrating manner. Since the light conductor mats 5 are moved transversely to their main direction of extension but are permeable to fluid at least in part, some of the nutrient solution 2 flows through the light conductor mat 5 when the mat is moved. This preferably produces swirling, and results in very good mixing of the nutrient solution and the phototrophic organisms surrounded thereby.

FIG. 5 is a schematic view of a photobioreactor system 100 according to an embodiment of the present. The photobioreactor system 100 comprises a photobioreactor 1 and a light source 27. In this case, the light source 27 may comprise one or more components for artificially generating light or for collecting naturally occurring light and then for coupling this light into a shared light guide 11 in order for it to be supplied to the photobioreactor 1.

The light source 27 may be designed as a light source 29 for collecting sunlight and coupling it into the light conducting fibres of the photobioreactor 1. Such a light source 29 may for example be designed as a solar collector 30 comprising a concave mirror which focuses sunlight onto a receiver. Additionally or alternatively, light conductor mats for absorbing sunlight in this sense may be considered a light source. The receiver may be connected to the light guide 11 in this case. In this way, when the sun is shining natural light can be used in a simple and energy-saving manner to illuminate the internal volume of the photobioreactor 1.

Alternatively or additionally, for this purpose, the light source 27 may be designed as a light source 31 for artificially generating light and coupling it into light conducting fibres of the photobioreactor 1. Such an artificial light source may for example be designed as an LED 32 or a laser 33 which radiates light onto an assembly 35 formed by a polariser and a screen, which assembly is in turn connected to the light guide 11 leading towards the photobioreactor 1.

The artificial light sources 32, 33 may be supplied with electrical power from alternative sources, such as by wind power 39 or by solar cells 41 or alternatively by conventional power 43. Here, the electrical power may be temporarily stored by a backup battery 37 for example, so that the artificial light source 31 can illuminate the photobioreactor 1 even when the sun is not shining.

A photodetector 45 is also provided in the photobioreactor system 100. This photodetector 45 is connected to light conducting fibres 9 of the at least one light conductor mat 5 in the photobioreactor 1 by means of the light guide 11, and is designed to detect light which for example has been emitted by the organisms contained in the nutrient solution 2 and has been coupled into the fibres 9 of the light conductor mat 5. On the basis of such detected light, conclusions can be drawn about vital functions of the phototrophic organisms from signals from the photodetector 45.

In summary, according to embodiments, a photobioreactor and a photobioreactor system are proposed in which one or more light conductor mats, in particular in the form of a woven fabric made up of light guides, are used to disperse light in a reactor. This means that light can also be supplied to deeper reactor layers. This allows there to be smaller surface area requirements for the photobioreactor, in particular compared with conventional open-pond systems. In addition, this allows high cell densities and a simple construction. Large volumes of nutrient solution to which organisms have been added may be illuminated with there being a low surface area. As a result, losses due to evaporation and the risk of contamination are minimised. Growth of the phototrophic organisms to be cultivated can be accelerated, in particular owing to the largely even illumination of the nutrient solution within the photobioreactor that is made possible.

In addition, the inside of the photobioreactor can be illuminated in a targeted manner with light of an appropriate wavelength, for example in a wavelength range of from 400 to 700 nm, preferably in a wavelength range of from 470 to 680 nm, in which algae growth is optimally promoted. By appropriately selecting the materials for the light conductor mat or by appropriately selecting the light sources, it is possible to couple as little infrared light as possible into the photobioreactor, so that said photobioreactor is not excessively heated and thus does not necessarily need to be actively cooled. In addition, the light can be radiated intermittently, for example at illumination durations of a few milliseconds, in order to thus increase the efficiency of the photosynthesis of the illuminated organisms and to accelerate growth of the organisms.

Furthermore, in addition to the option of evenly illuminating the inside of the container of the photobioreactor, the light conductor mats may also be used to thoroughly mix the nutrient solution received or contained therein in a targeted manner by said solution being moved by means of a mat-moving device within the nutrient solution.

In addition, in the proposed photobioreactor system, another photodetector may be provided which is connected to fibres of the light conductor mat, in order to thus allow on-site monitoring of vital functions of the organisms to be cultivated, by detecting the light signals emitted therefrom by means of the light conducting fibres that are present anyway. Here, light and/or signals are transduced in both directions of the light conducting fibres in accordance with transmit/receive modes.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the embodiment in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the embodiment as set forth in the appended claims and their legal equivalents.

1. A photobioreactor for cultivating phototrophic organisms, the photobioreactor comprising:
a container for receiving the phototrophic organisms together with a nutrient solution; and
at least one laterally light out-coupling light conductor mat;
wherein the light conductor mat is arranged within the container, and
wherein the light conductor mat comprises a plurality of light conducting fibres, that are arranged and/or designed such that light that is coupled into the fibres at one end of a fibre leaves the fibres laterally at least in part.

2. The photobioreactor of claim 1, wherein the light conductor mat is arranged such that a minimum distance between a position in the container and the closest region of the light conductor mat is less than 10 cm for at least 90% of the possible positions within the container.

3. The photobioreactor of claim 1, wherein the container has dimensions of greater than 50 cm in all directions in space.

4. The photobioreactor of claim 1, comprising a plurality of laterally light out-coupling light conductor mats, that are distributed over the entire volume of the container.

5. The photobioreactor of claim 1, wherein the light conducting fibres are arranged in the light conductor mat so as to be locally bent such that portions of light guided in a fibre are locally coupled out of the fibre laterally, at least in regions having a minimum radius of curvature.

6. The photobioreactor of claim 1, wherein the light conducting fibres are woven in the light conductor mat.

7. The photobioreactor of claim 1, wherein the light conducting fibres have local variations in the index of refraction.

8. The photobioreactor of claim 1, wherein scattering centres and/or fluorescence centres are integrated in the light conducting fibres.

9. The photobioreactor of claim 1, wherein the light conducting fibres are made of a material that does not substantially transmit light in the infrared wavelength range.

10. The photobioreactor of claim 1, further comprising a mat moving device, that moves the light conductor mat relative to the container.

11. A photobioreactor system, comprising:
a photobioreactor for cultivating phototrophic organisms, the photobioreactor comprising:
a container for receiving the phototrophic organisms can be received together with a nutrient solution; and
at least one laterally light out-coupling light conductor mat;
wherein the light conductor mat is arranged within the container, and
wherein the light conductor mat comprises a plurality of light conducting fibres, that are arranged and/or designed such that light that is coupled into the fibres at one end of a fibre leaves the fibres laterally at least in part; and
a light source,
wherein the light source is coupled to light conducting fibres of the at least one light conductor mat of the photobioreactor in order to couple light from the light source into the light conducting fibres.

12. The photobioreactor system of claim 11, wherein the light source collects sunlight and couples it into the light conducting fibres.

13. The photobioreactor system of claim 11, wherein the light source artificially generates light and couples it into the light conducting fibres.

14. The photobioreactor system of claim 11, wherein the light source only couples light that is substantially within a wavelength range from 400 to 700 nm into the light conducting fibres.

15. The photobioreactor system of claim 11, further comprising a photodetector, wherein the photodetector is connected to light conducting fibres of the at least one light conductor mat of the photobioreactor in order to collect light which has been coupled into the light conducting fibres from the inside of the container of the photobioreactor.

16. A photobioreactor for cultivating phototrophic organisms, the photobioreactor comprising:
a container for receiving the phototrophic organisms can be received together with a nutrient solution; and
at least one laterally light out-coupling light conductor mat;
wherein the light conductor mat is arranged within the container, and
wherein the light conductor mat comprises a plurality of light conducting fibres, that are arranged and/or designed such that light that is coupled into the fibres at one end of a fibre leaves the fibres laterally at least in part,
wherein the light conductor mat is arranged such that a minimum distance between a position in the container and the closest region of the light conductor mat is less than 10 cm for at least 90% of the possible positions within the container; and
wherein the container has dimensions of greater than 50 cm in all directions in space.

17. The photobioreactor of claim 16, further comprising:
a plurality of laterally light out-coupling light conductor mats that are distributed over the entire volume of the container,
wherein the light conducting fibres are arranged in the light conductor mat so as to be locally bent such that portions of light guided in a fibre are locally coupled out of the fibre laterally, at least in regions having a minimum radius of curvature.

18. The photobioreactor of claim 17, wherein the light conducting fibres are woven in the light conductor mat,
wherein the light conducting fibres have local variations in the index of refraction, and
wherein scattering centres and/or fluorescence centres are integrated in the light conducting fibres.

19. The photobioreactor of claim 18, wherein the light conducting fibres are made of a material that does not substantially transmit light in the infrared wavelength range and further comprising a mat moving device that moves the light conductor mat relative to the container.
